SEQUENCE LISTING (4 sequences)

SEQ ID NO: 1 - DNA, 975 bp, Homo sapiens
  CDS (105)..(563)
cctggccggaaactgagccgttcacttccgccaccagccggaagttttctgtcactggacgccaaggagttttcggtggctcagctgggtaaccggggatcaccatggcggcctcattggtggggaagaagatcgtgtttgtaacggggaacgccaagaagctggaggaggtacaggggcccgtgctggttgaggacacttgtctgtgcttcaatgcccttggagggctccccggcccctacataaagtggtttctggagaagttaaagcctgaaggtctccaccagctcctggccgggttcgaggacaagtcagcctatgcgctctgcacgtttgcactcagcaccggggacccaagccagcccgtgcgcctgttcaggggccggacctcgggccggatcgtggcacccagaggctgccaggactttggctgggacccctgctttcagcctgatggatatgagcagacgtacgcagagatgcctaaggcggagaagaacgctgtctcccatcgcttccgggccctgctggagctgcaggagtactttggcagtttggcagcttgacttctgcagctggaggaggcccctcaggccggggatctggggagggctagcccaaaacctcccgcatcgggcaggcaccccctgaagtacttccttcagggtttcccctttgtgagggtgtcaagtagcctcaccggcctgtctggaggagcagctggctctgctctgagaaactctggcaagtggacgccattctcttgcccttaggattcactgctctctcctacagccgccaggcctggggtcctgaaaggaccttgggtggtaaagctgtacttggtgggagtgagggcgtggggaggaaccatgcaaatcgccttccatggtttttaaatgcagtaaataacatttctggatgagacttgtttccaaaataaaccagctatatctgttccgaaaaaaaaaaaaa

SEQ ID NO: 2 - protein, 153 aa, Homo sapiens
MAASLVGKKIVFVTGNAKKLEEVQGPVLVEDTCLCFNALGGLPGPYIKWFLEKLKPEGLHQLLAGFEDKSAYALCTFALSTGDPSQPVRLFRGRTSGRIVAPRGCQDFGWDPCFQPDGYEQTYAEMPKAEKNAVSHRFRALLELQEYFGSLAA

SEQ ID NO: 3 - DNA, 1047 bp, Homo sapiens
  CDS (105)..(635)
cctggccggaaactgagccgttcacttccgccaccagccggaagttttctgtcactggacgccaaggagttttcggtggctcagctgggtaaccggggatcaccatggcggcctcattggtcgttcagattctaggagataagtttccatgcactttggtggcacagaaaattgacctgccggagtaccagggggagccggatgagatttccatacagaaatgtcaggaggcagttcgccaggtacaggggcccgtgctggttgaggacacttgtctgtgcttcaatgcccttggagggctccccggcccctacataaagtggtttctggagaagttaaagcctgaaggtctccaccagctcctggccgggttcgaggacaagtcagcctatgcgctctgcacgtttgcactcagcaccggggacccaagccagcccgtgcgcctgttcaggggccggacctcgggccggatcgtggcacccagaggctgccaggactttggctgggacccctgctttcagcctgatggatatgagcagacgtacgcagagatgcctaaggcggagaagaacgctgtctcccatcgcttccgggccctgctggagctgcaggagtactttggcagtttggcagcttgacttctgcagctggaggaggcccctcaggccggggatctggggagggctagcccaaaacctcccgcatcgggcaggcaccccctgaagtacttccttcagggtttcccctttgtgagggtgtcaagtagcctcaccggcctgtctggaggagcagctggctctgctctgagaaactctggcaagtggacgccattctcttgcccttaggattcactgctctctcctacagccgccaggcctggggtcctgaaaggaccttgggtggtaaagctgtacttggtgggagtgagggcgtggggaggaaccatgcaaatcgccttccatggtttttaaatgcagtaaataacatttctggatgagacttgtttccaaaataaaccagctatatctgttccgaaaaaaaaaaaaa

SEQ ID NO: 4 - protein, 177 aa, Homo sapiens
MAASLVVQILGDKFPCTLVAQKIDLPEYQGEPDEISIQKCQEAVRQVQGPVLVEDTCLCFNALGGLPGPYIKWFLEKLKPEGLHQLLAGFEDKSAYALCTFALSTGDPSQPVRLFRGRTSGRIVAPRGCQDFGWDPCFQPDGYEQTYAEMPKAEKNAVSHRFRALLELQEYFGSLAA

(19) United States
(12) Patent Application Publication
(10) Pub. No.: US 2005/0266446 A1
(43) Pub. Date: Dec. 1, 2005

(54) Human ITPase-related gene variants associated with lung cancers

(76) Inventor: Ken-Shwo Dai, Hsinchu (TW)

Correspondence Address: LADAS & PARRY, 26 WEST 61ST STREET, NEW YORK, NY, 10023, US

(21) Appl. No.: 11/098,765

(22) Filed: Apr. 4, 2005

Related U.S. Application Data
(62) Division of application No. 10/103,335, filed on Mar. 21, 2002.

(51) Int. Cl. C12Q 1/68; G01N 33/574; C07K 14/82; C07H 21/04; C12P 21/06

(52) U.S. Cl. 435/6; 435/7.23; 435/69.1; 435/320.1; 435/325; 530/350; 536/23.5

(57) ABSTRACT

The invention relates to the nucleic acid sequences of two novel human ITPase-related gene variants (ITPA1 and ITPA2) and the polypeptides encoded thereby. The invention also relates to the process for producing the polypeptides encoded by the ITPA1 and ITPA2. The invention further relates to the use of the nucleic acids of the ITPA1 and ITPA2 and the polypeptides encoded thereby in diagnosing diseases associated with the deficiency of human ITPase gene, in particular lung cancers.

### FIELD OF THE INVENTION

The invention relates to the nucleic acid sequences of two novel human ITPase-related gene variants (ITPA1 and ITPA2), the polypeptides encoded thereby, the preparation process thereof, and the uses of the same in diagnosing diseases associated with the deficiency of human ITPase gene, in particular, lung cancers.

### BACKGROUND OF THE INVENTION

Lung cancer is one of the major causers of cancer-related deaths in the world. There are two primary types of lung cancers: small cell lung cancer (SCLC) and non-small cell lung cancer (NSCLC) (Carney, (1992a) Curr. Opin. Oncol. 4:292-8). Small cell lung cancer accounts for approximately 25% of lung cancer and spreads aggressively (Smyth et al. (1986) Q J Med. 61: 969-76; Carney, (1992b) Lancet 339: 843-6). Non-small cell lung cancer represents the majority (about 75%) of lung cancer and is further divided into three main subtypes: squamous cell carcinoma, adenocarcinoma, and large cell carcinoma (Ihde and Minutesna, (1991) Cancer 15: 105-54). In recent years, much progress has been made toward understanding the molecular and cellular biology of lung cancers. Many important contributions have been made by the identification of several key genetic factors associated with lung cancers. However, the treatments of lung cancers still mainly depend on surgery, chemotherapy, and radiotherapy. This is because the molecular mechanisms underlying the pathogenesis of lung cancers remain largely unclear.

A recent hypothesis suggests that lung cancer is caused by genetic mutations of at least 10 to 20 genes (Sethi, (1997) BMJ. 314: 652-655). Therefore, future strategies for the prevention and treatment of lung cancers will be focused on the elucidation of these genetic substrates, in particular, the genes associated with pathogenesis induced by tobacco carcinogen. A previous study has shown that plasma level of leukotriene B4 (LTB4) was increased by the presence of a tobacco carcinogen NNK (Castonguay et al. (1998) Exp Lung Res 24:605-15). LTB4 is a potent activator of inosine triphosphate pyrophosphatase (ITPase) which could effectively activate ITP hydrolysis (Klinker and Seifert, (1997) Biochem Pharmacol 54:551-62). Recently, the human ITPase has been cloned and mapped on chromosome 20p (Lin et al. (2001) J Boil Chem 276:18695-701), a region associated with lung cancer (Michelland et al. (1999) Cancer Genet Cytogenet 114:22-30). ITP, has been mentioned to be a mutational risk factor for the cell (Galperin, (2001) Comp Funct Genom 2: 14-18). In addition, the cytotoxic effect of ITP has been shown to be associated with chromosome aberration rate, the mitotic rate, sister-chromatid exchange (SCE) frequency, and the proportion of first (X1), second (X2) and third (X3) division metaphases (Vormittag and Brannath, (2001) Mutat Res 476:71-81). Taken together with the chromosomal localization of ITPase (a region associated with lung cancer), we believe that the discovery of gene variants of ITPase (an essential enzyme for ITP hydrolysis) (Hwang et al. (1999) Nat Struct Biol 6:691-6), may be important targets for diagnostic markers of lung cancers.

### SUMMARY OF THE INVENTION

The invention provides two ITPase-related gene variants found in human lung cancer tissues and the polypeptide sequences encoded thereby, which are useful in the diagnosis of the diseases associated with these deficiency of human ITPase gene, in particular lung cancers, preferably SCLC and NSCLC.

The invention further provides an expression vector and host cell for expressing ITPA1 and ITPA2.

The invention further provides a method for producing the polypeptides encoded by ITPA1 and ITPA2.

The invention further provides an antibody specifically binding to the polypeptides encoded by ITPA1 and ITPA2.

The invention also provides methods for diagnosing diseases associated with the deficiency of human ITPase gene, in particular lung cancers, preferably SCLC and NSCLC.

### BRIEF DESCRIPTION OF THE DRAWINGS

FIG. 1A to 1B show the nucleic acid sequence of ITPA1 (SEQ ID NO:1) and the amino acid sequence encoded thereby (SEQ ID NO:2).

FIG. 2A to 2C show the nucleic acid sequence of ITPA2 (SEQ ID NO:3) and the amino acid sequence encoded thereby (SEQ ID NO:4).

FIG. 3A to 3G show the nucleotide sequence alignment between human ITPase gene and ITPA1 and ITPA2.

FIG. 4A to 4B show the amino acid sequence alignment among human ITPase and the polypeptides encoded by ITPA1 and ITPA2.

### DETAILED DESCRIPTION OF THE INVENTION

According to the invention, all technical and scientific terms used have the same meanings as commonly understood by persons skilled in the art.

The term “antibody,” as used herein, denotes intact molecules (a polypeptide or group of polypeptides) as well as fragments thereof, such as Fab, R(ab′)₂, and Fv fragments, which are capable of binding the epitopic determinutesant. Antibodies are produced by specialized B cells after stimulation by an antigen. Structurally, antibody consists of four subunits including two heavy chains and two light chains. The internal surface shape and charge distribution of the antibody binding domain are complementary to the features of an antigen. Thus, antibody can specifically act against the antigen in an immune response.

The term “base pair (bp),” as used herein, denotes nucleotides composed of a purine on one strand of DNA which can be hydrogen bonded to a pyrimidine on the other strand. Thymine (or uracil) and adenine residues are linked by two hydrogen bonds. Cytosine and guanine residues are linked by three hydrogen bonds.

The term “Basic Local Alignment Search Tool (BLAST; Altschul et al., (1997) Nucleic Acids Res. 25: 3389-3402),” as used herein, denotes programs for evaluation of homologies between a query sequence (amino or nucleic acid) and a test sequence as described by Altschul et al. (Nucleic Acids Res. 25: 3389-3402, 1997). Specific BLAST programs are described as follows:

(1) BLASTN compares a nucleotide query sequence against a nucleotide sequence database;

(2) BLASTP compares an amino acid query sequence against a protein sequence database;

(3) BLASTX compares the six-frame conceptual translation products of a query nucleotide sequence against a protein sequence database;

(4) TBLASTN compares a query protein sequence against a nucleotide sequence database translated in all six reading frames; and

(5) TBLASTX compares the six-frame translations of a nucleotide query sequence against the six-frame translations of a nucleotide sequence database.

The term “cDNA,” as used herein, denotes nucleic acids that synthesized from a mRNA template using reverse transcriptase.

The term “cDNA library,” as used herein, denotes a library composed of complementary DNAs which are reverse-transcribed from niRNAs.

The term “complement,” as used herein, denotes a polynucleotide sequence capable of forming base pairing with another polynucleotide sequence. For example, the sequence 5′-ATGGACTTACT-3′ binds to the complementary sequence 5′-AGTAAGTCCAT-3′.

The term “deletion,” as used herein, denotes a removal of a portion of one or more amino acid residues/nucleotides from a gene.

The term “expressed sequence tags (ESTs),” as used herein, denotes short (200 to 500 base pairs) nucleotide sequence that derives from either 5′ or 3′ end of a cDNA.

The term “expression vector,” as used herein, denotes nucleic acid constructs which contain a cloning site for introducing the DNA into vector, one or more selectable markers for selecting vectors containing the DNA, an origin of replication for replicating the vector whenever the host cell divides, a terminator sequence, a polyadenylation signal, and a suitable control sequence which can effectively express the DNA in a suitable host. The suitable control sequence may include promoter, enhancer and other regulatory sequences necessary for directing polymerases to transcribe the DNA.

The term “host cell,” as used herein, denotes a cell which is used to receive, maintain, and allow the reproduction of an expression vector comprising DNA. Host cells are transformed or transfected with suitable vectors constructed using recombinant DNA methods. The recombinant DNA introduced with the vector is replicated whenever the cell divides.

The term “insertion” or “addition,” as used herein, denotes the addition of a portion of one or more amino acid residues/nucleotides to a gene.

The term “in silico,” as used herein, denotes a process of using computational methods (e.g., BLAST) to analyze DNA sequences.

The term “polymerase chain reaction (PCR),” as used herein, denotes a method which increases the copy number of a nucleic acid sequence using a DNA polymerase and a set of primers (about 20 bp oligonucleotides complementary to each strand of DNA) under suitable conditions (successive rounds of primer annealing, strand elongation, and dissociation).

The term “protein” or “polypeptide,” as used herein, denotes a sequence of amino acids in a specific order that can be encoded by a gene or by a recombinant DNA. It can also be chemically synthesized.

The term “nucleic acid sequence” or “polynucleotide,” as used herein, denotes a sequence of nucleotide (guanine, cytosine, thymine or adenine) in a specific order that can be a natural or synthesized fragment of DNA or RNA. It may be single-stranded or double-stranded.

The term “reverse transcriptase-polymerase chain reaction (RT-PCR),” as used herein, denotes a process which transcribes MRNA to complementary DNA strand using reverse transcriptase followed by polymerase chain reaction to amplify the specific fragment of DNA sequences.

The term “transformation,” as used herein, denotes a process describing the uptake, incorporation, and expression of exogenous DNA by prokaryotic host cells.

The term “transfection,” as used herein, a process describing the uptake, incorporation, and expression of exogenous DNA by eukaryotic host cells.

The term “variant,” as used herein, denotes a fragment of sequence (nucleotide or amino acid) inserted or deleted by one or more nucleotides/amino acids.

In the first aspect, the invention provides the polypeptide encoded by two novel human ITPase-related gene variants (ITPA1 and ITPA2) and the fragments thereof, as well as the nucleotide sequences of ITPA1 and ITPA2.

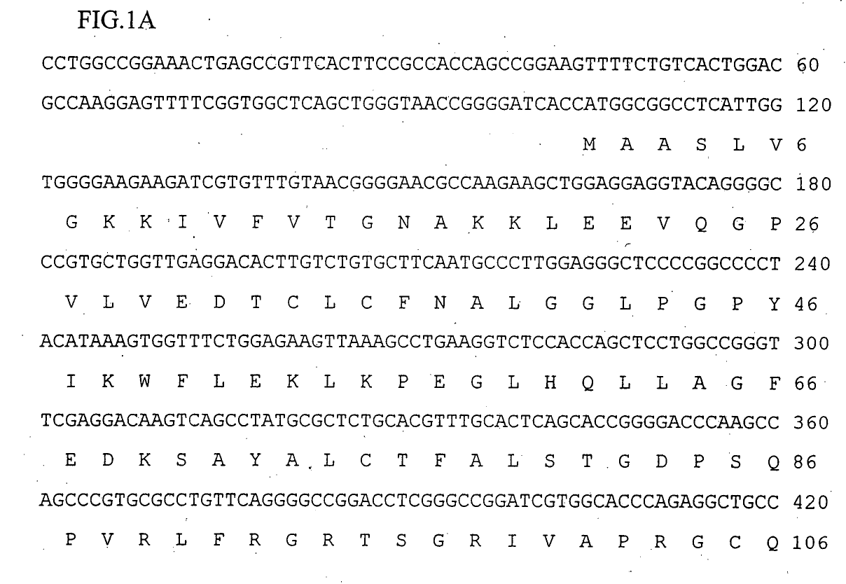

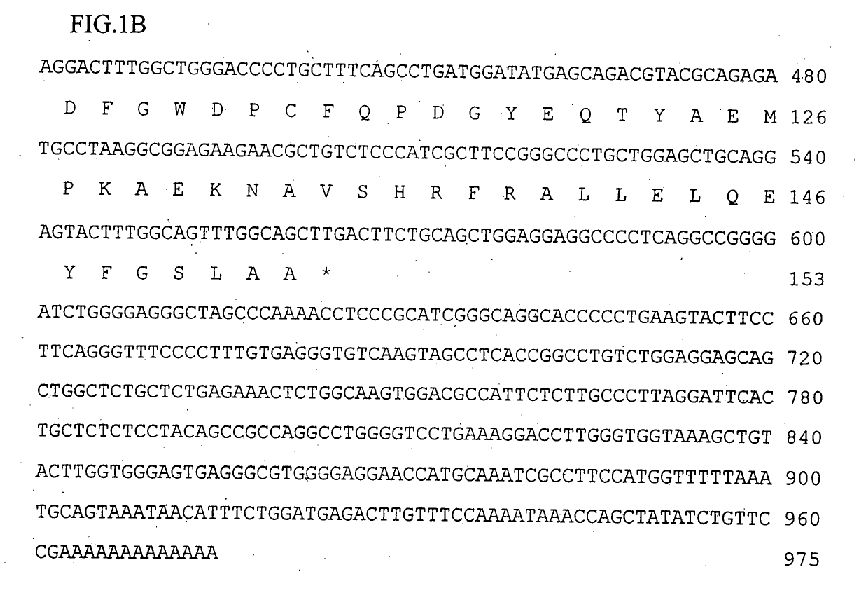

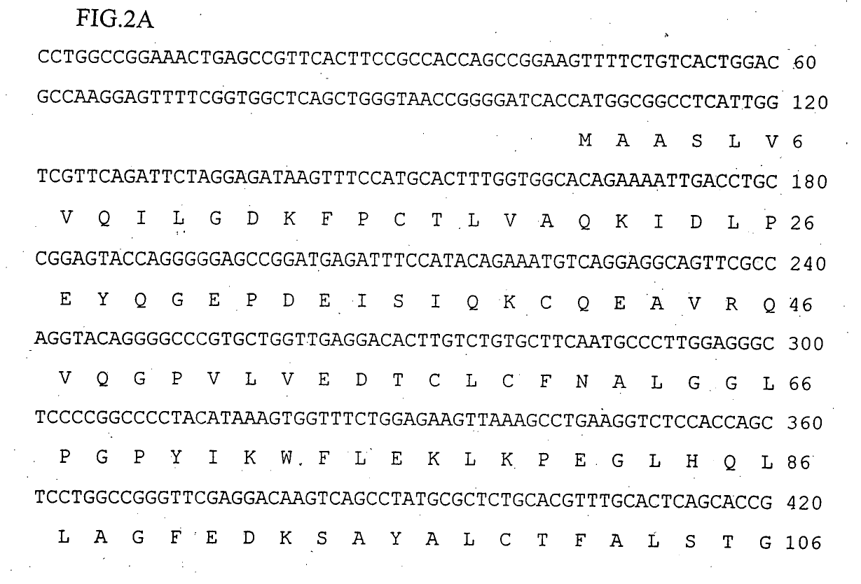

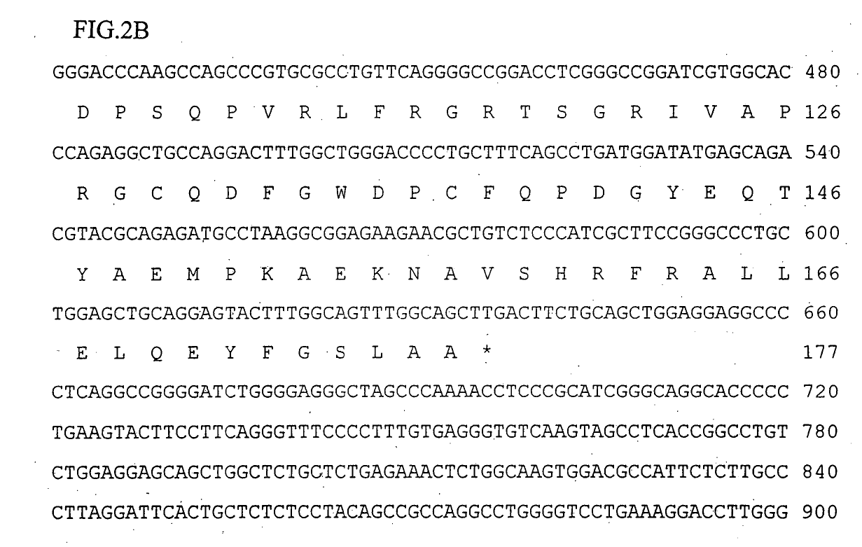

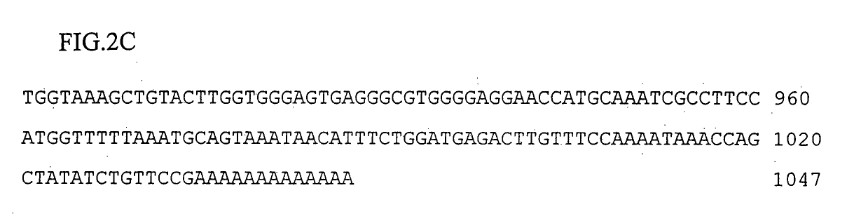

According to the invention, human ITPase cDNA sequence was used to query the human lung EST databases (a normal lung, a large cell lung cancer, and a small cell lung cancer) using BLAST program to search for ITPase-related gene variants. Two human cDNA partial sequences (i.e., ESTs) deposited in the databases showing similarity to ITPA were isolated and sequenced. These clones (named ITPA1 and ITPA2) were isolated from small cell lung cancer and large cell lung cancer cDNA libraries, respectively. FIGS. 1 and 2 show the nucleic acid sequences (SEQ ID NOs:1, and 3) of the variants (ITPA1 and ITPA2) and corresponding amino acid sequences (SEQ ID NOs:2, and 4) encoded thereby.

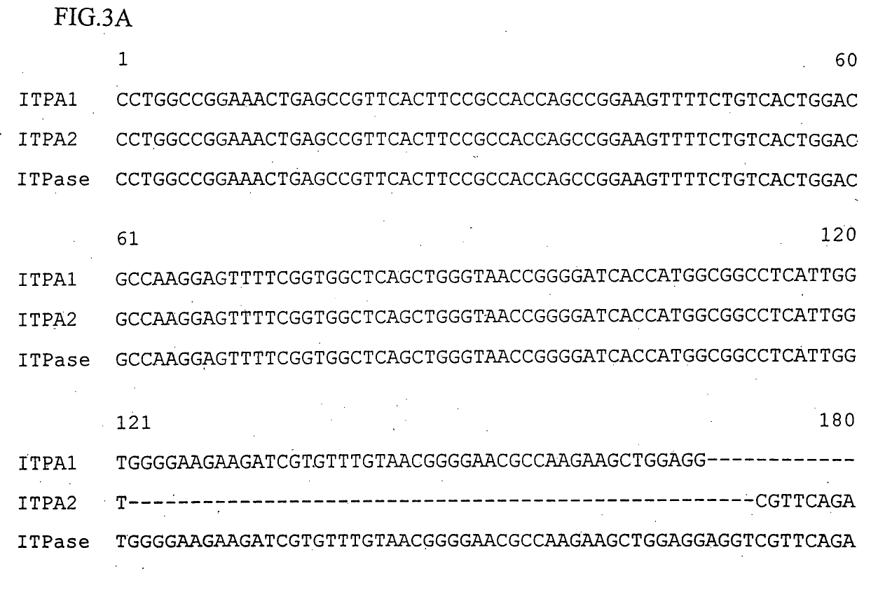

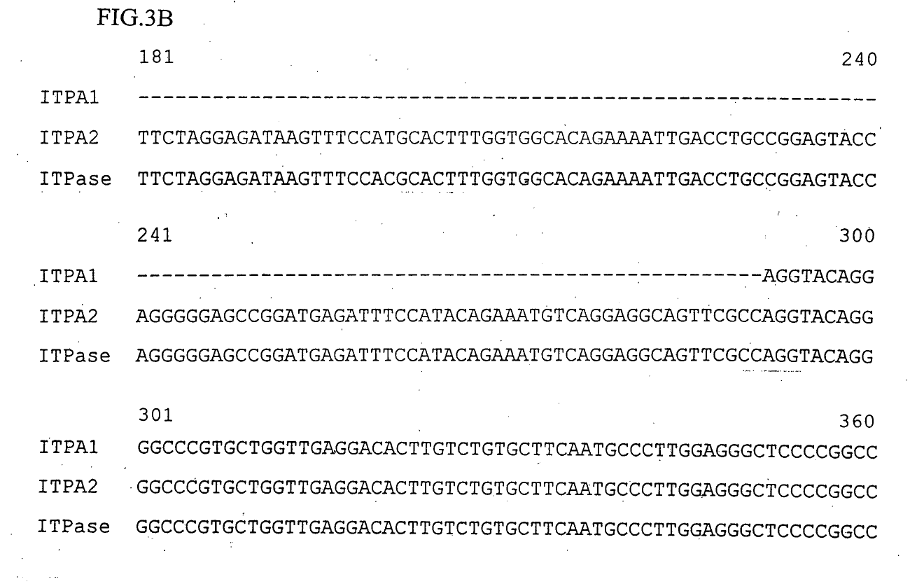

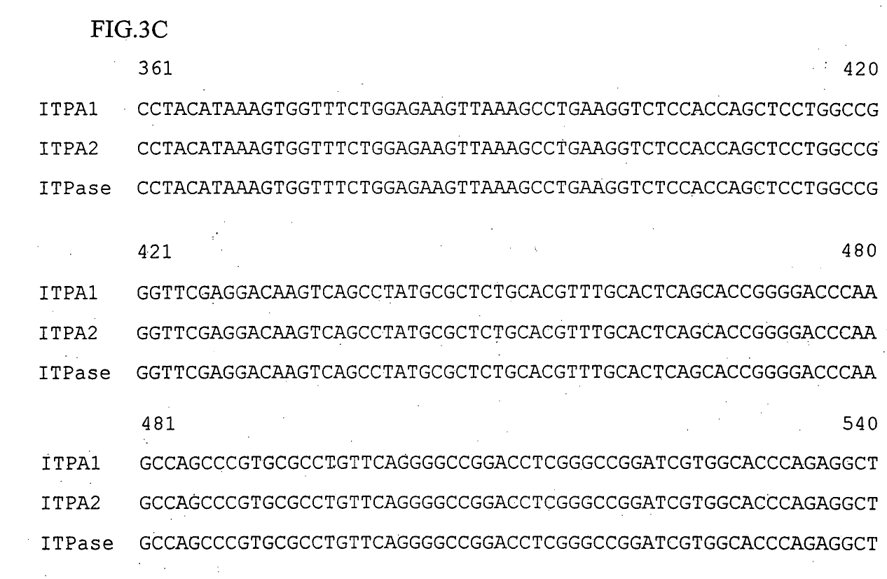

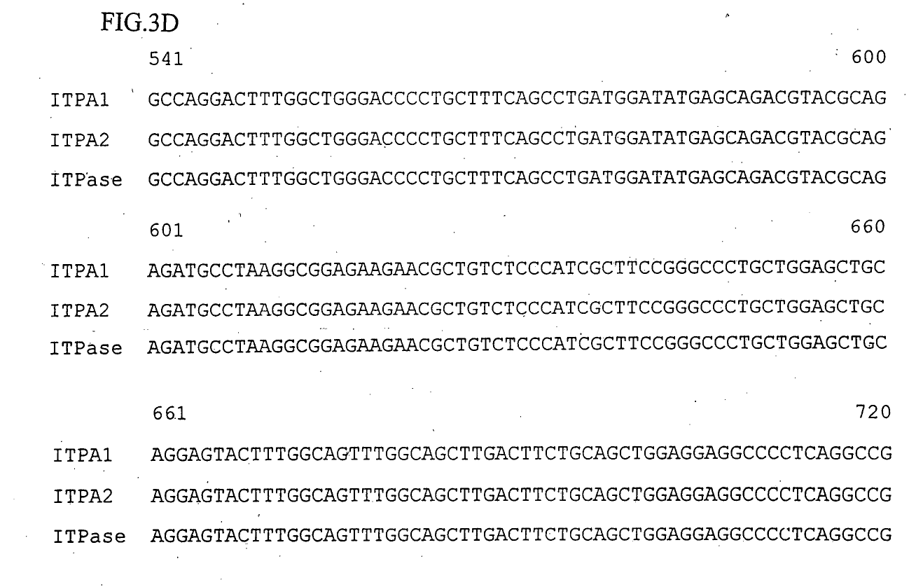

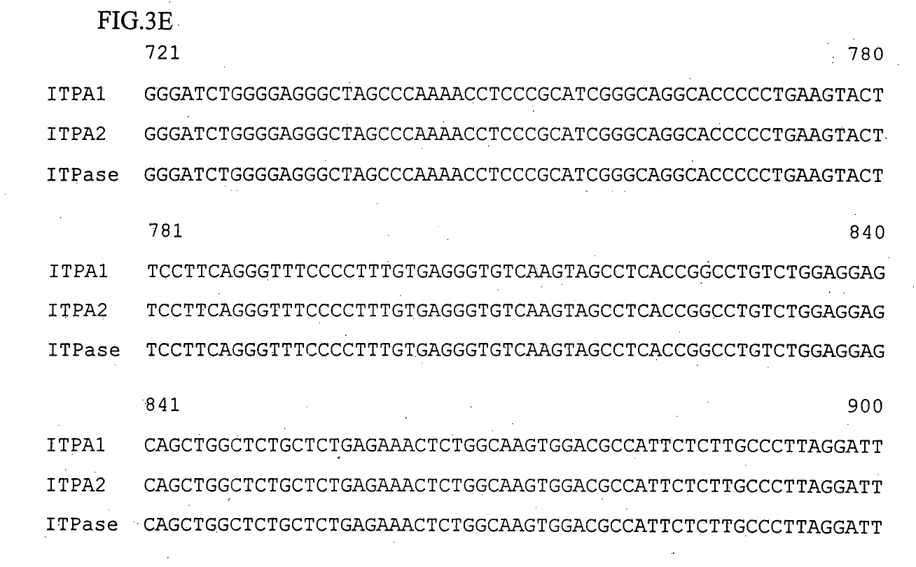

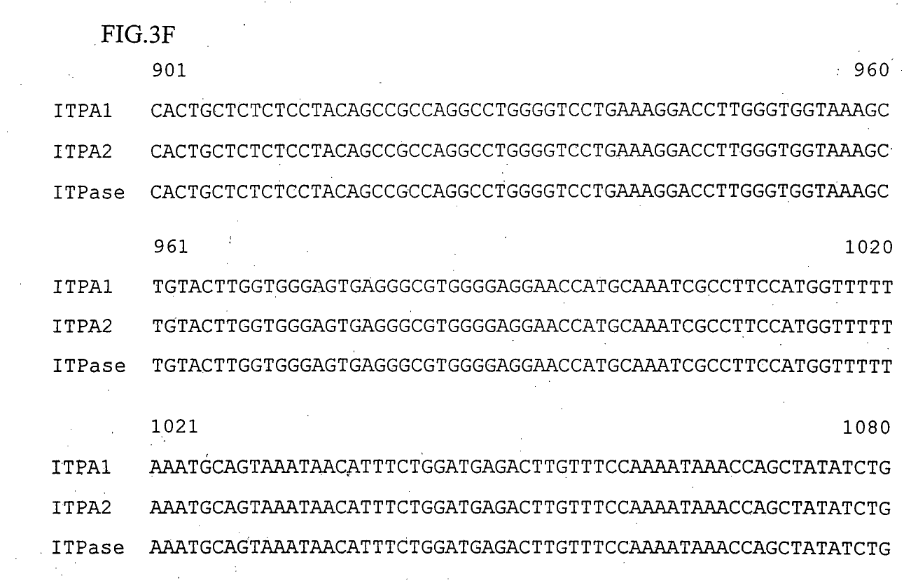

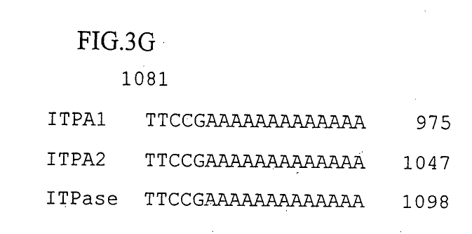

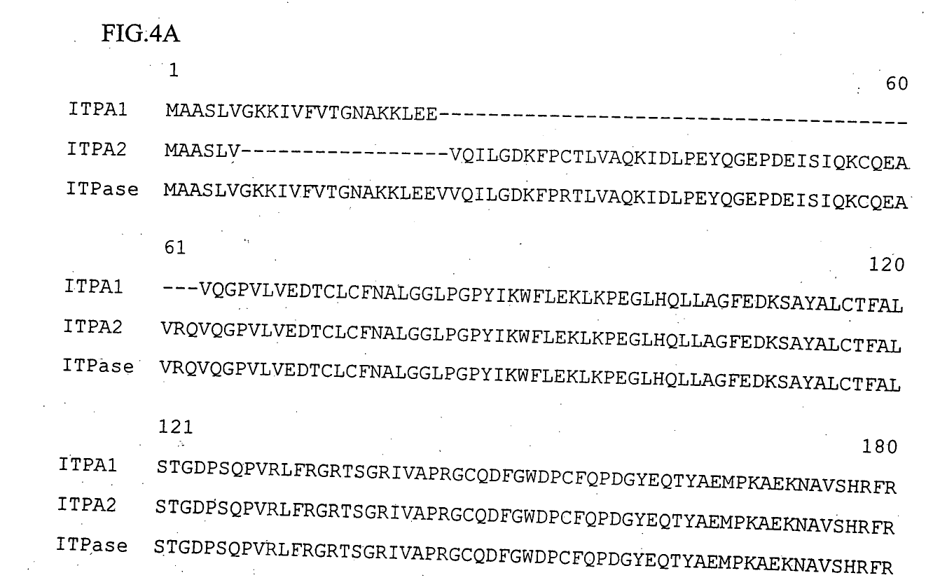

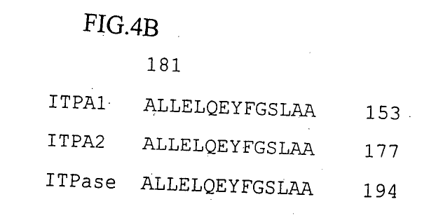

The full-length of the ITPA1 cDNA is a 975 bp clone containing a 459 bp open reading frame (ORF) extending from 105 bp to 563 bp, which corresponds to an encoded protein of 153 amino acid residues with a predicted molecular mass of 16.8 kDa. The full-length of the ITPA2 cDNA is a 1047 bp clone containing a 531 bp ORF extending from 105 bp to 635 bp, which corresponds to an encoded protein of 177 amino acid residues with a predicted molecular mass of 19.6 kDa. The sequences around the initiation to ATG codon of ITPA1 and ITPA2 (located at nucleotide 105 to 107 bp) were matched with the Kozak consensus sequence (A/GCCATGG) (Kozak, (1987) Nucleic Acids Res. 15: 8125-48; Kozak, (1991) J Cell Biol. 115: 887-903.). To determine the variations (insertion/deletion) in sequences of ITPA1 and ITPA2 cDNA clones, an alignment of ITPase nucleotide/amino acid sequence with these clones was performed (FIGS. 3 and 4). Two major genetic deletions were found in the aligned sequences. ITPA1 is an in-frame 123 bp deletion (encoding 41 amino acids) in the sequence of ITPase from 169 to 291 bp and ITPA2 is an in-frame 51 bp deletion (encoding 17 amino acids) in the sequence of ITPase from 122 to 172 bp.

In the invention, a search of ESTs deposited in dbEST (Boguski et al., (1993) Nat Genet. 4: 332-3) at NCBI (National Center for Biotechnology Information) was performed. Two ESTs were found to confirm the missing region described in ITPA1 and TPA2. One EST (GenBank accession number BI115811), confirmed the absence of 123 bp region on ITPA1 nucleotide sequence, was found to be isolated from a SCLC cDNA library. Another EST (GenBank accession number BG332818), confirmed the absence of 51 bp region on ITPA2 nucleotide sequence, was found to be isolated from a large cell lung cancer cDNA library. This suggests that the absence of both 123 bp and 51 bp deletion segments may serve as useful markers for diagnosing SCLC and large cell lung cancer, respectively. Therefore, any nucleotide fragments comprising nucleotides 168 to 169 of ITPA1 and nucleotides 122 to 123 of ITPA2 may be used as probes for determining the presence of ITPA1 and ITPA2 under highly stringent conditions. An alternative approach is that any set of primers for amplifying the fragment containing nucleotides 168 to 169 of ITPA1 and nucleotides 122 to 123 of ITPA2 may be used for determining the presence of the variants.

Scanning the ITPA1 sequence against the profile entries in PROSITE (ScanProsite) indicated that ITPA1 protein contains two protein kinase C phosphorylation sites (96-98aa and 135-137aa), two casein kinase II phosphorylation sites (80-83aa, and 122-125aa), one tyrosine kinase phosphorylation site (139-147aa), and one amidation site (6-9aa). Scanning the ITPA2 sequence against the profile entries in PROSITE (ScanProsite) indicated that ITPA2 protein contains two protein kinase C phosphorylation sites (120-122aa and 159-161aa), two casein kinase II phosphorylation sites (104-107aa, and 146-149aa), and one tyrosine kinase phosphorylation site (163-171aa).

According to the invention, the polypeptides encoded by ITPA1 and ITPA2 and the fragments thereof may be produced through genetic engineering techniques. For instance, they may be produced by using appropriate host cells that have been transformed with recombinant DNAs that code for the desired polypeptides or fragments thereof. The nucleotide sequence of ITPA1 and ITPA2 or the fragment thereof is inserted into an appropriate expression vector, i.e., a vector which contains the necessary elements for the transcription and translation of the inserted coding sequence in a suitable host. The nucleotide sequence is inserted into the vector in a manner such that it will be expressed under appropriate conditions (e.g., in proper orientation and correct reading frame and with appropriate expression sequences, including an RNA polymerase binding sequence and a ribosomal binding sequence).

Any method that is known to those skilled in the art may be used to construct expression vectors containing the sequences of ITPA1 or ITPA2 and appropriate transcriptional/translational control elements. These methods may include in vitro recombinant DNA and synthetic techniques, and in vivo genetic recombinant techniques (see, e.g., Sambrook, J. Cold Spring Harbor Press, Plainview N.Y., ch. 4, 8, and 16-17; Ausubel, R. M. et al. (1995) Current protocols in Molecular Biology, John Wiley & Sons, New York N.Y., ch. 9, 13, and 16.).

A variety of expression vector/host systems may be utilized to express ITPA1 and ITPA2. These include, but not limited to, microorganisms such as bacteria transformed with recombinant bacteriophage, plasmid, or cosmid DNA expression vector; yeast transformed with yeast expression vector; insect cell systems infected with virus (e.g., baculovirus); plant cell system transformed with viral expression vector (e.g., cauliflower mosaic virus, CaMV, or tobacco mosaic virus, TMV); or animal cell system infected with virus (e.g., vaccina virus, adenovirus, etc.). Preferably, the host cell is a bacterium, and more preferably, the bacterium is E. coli.

Alternatively, the polypeptides encoded by ITPA1 and ITPA2 or fragments thereof may be synthesized using chemical methods. For example, peptide synthesis can be performed using various solid-phase techniques (Roberge, J. Y. et al. (1995) Science 269: 202 to 204). Automated synthesis may be achieved using the ABI 431A peptide synthesizer (Perkin-Elmer).

According to the invention, the fragments of the nucleotide sequences of ITPA1 and ITPA2 and the polypeptides encoded thereby are used as primers or probes and immunogens, respectively. Preferably, the purified fragments of the human ITPA1 and ITPA2 are used. The fragments may be produced by enzyme digestion, chemical cleavage of isolated or purified polypeptide or nucleic acid sequences, or chemical synthesis; and then further isolated or purified. Such isolated or purified fragments of the polypeptides and nucleic acid sequences can be used directly as immunogens and primers or probes, respectively.

The invention further provides the antibodies which specifically bind one or more out-surface epitopes of the polypeptides encoded by ITPA1 and ITPA2.

According to the invention, the immunization of mammals with immunogens described herein, preferably humans, rabbits, rats, mice, sheep, goats, cows, or horses, is performed by the procedures well known to those skilled in the art, for the purpose of obtaining antisera containing polyclonal antibodies or hybridoma lines secreting monoclonal antibodies.

Monoclonal antibodies can be prepared by standard techniques, given the teachings contained herein. Such techniques are disclosed, for example, in U.S. Pat. Nos. 4,271,145 and 4,196,265. Briefly, an animal is immunized with the immunogen. Hybridomas are prepared by fusing spleen cells from the immunized animal with myeloma cells. The fusion products are screened for those producing antibodies that bind to the immunogen. The positive hybridoma clones are isolated, and the monoclonal antibodies are recovered from those clones.

Immunization regimens for production of both polyclonal and monoclonal antibodies are well-known in the art. The immunogen may be injected by any of a number of routes, including subcutaneous, intravenous, intraperitoneal, intradermal, intramuscular, mucosal, or a combination thereof. The immunogen may be injected in soluble form, aggregate form, attached to a physical carrier, or mixed with an adjuvant, using methods and materials well-known in the art. The antisera and antibodies may be purified using column chromatography methods well known to those skilled in the art.

According to the invention, antibody fragments which contain specific binding sites for the polypeptides or fragments thereof may also be generated. For example, such fragments include, but are not limited to, F(ab′)₂ fragments produced by pepsin digestion of the antibody molecule and Fab fragments generated by reducing the disulfide bridges of the F(ab′)₂ fragments.

Many gene variants have been found to be associated with diseases (Stallings-Mann et al., (1996) Proc Natl Acad Sci USA 93: 12394-9; Liu et al., (1997) Nat Genet 16:328-9; Siffert et al., (1998) Nat Genet 18: 45 to 8; Lukas et al., (2001) Cancer Res 61: 3212 to 9). Since ITPase is associated with an aberrant chromosomal region (chromosome 20p) found in lung cancer, it suggests that the ITPA1 and ITPA2 which have genetic deletion of nucleotide/amino acid sequences may result in lung cancer development and may serve as markers for the diagnosis of diseases associated with the deficiency of human ITPase gene, in particular lung cancers, e.g., SCLC and large cell lung cancer. Based on the. cDNA libraries of the matched ESTs, ITPA1 can be specifically associated with SCLC whereas ITPA2 can be associated with large cell lung cancer. Thus, the expression level of ITPA1 and ITPA2 relative to ITPase may be a useful indicator for screening of patients suspected of having lung cancers or more specifically the SCLC or large cell lung cancer. This suggests that the index of relative expression level (mRNA or protein) may confer an increased susceptibility to the same.

Accordingly, the invention further provides methods for diagnosing the diseases associated with the deficiency of ITPase gene in a mammal, in particular lung cancers, preferably, SCLC and NSCLC.

The method for diagnosing the diseases associated with the deficiency of human ITPase gene may be performed by detecting the nucleotide sequence of the ITPA1 and ITPA2 of the invention, which comprises the steps of: (1) extracting the total RNA of cells obtained from the mammal; (2) amplifying the RNA by reverse transcriptase-polymerase chain reaction (RT-PCR) with a set of primers to obtain a cDNA comprising the fragments comprising nucleotides 168 through 173 of SEQ ID NO: 1 or nucleotides 120 through 125 of SEQ ID NO: 3; and (3) detecting whether the cDNA is obtained. If necessary, the amount of the obtained cDNA sample may be determined.

In this embodiment, one of the primers may be designed to have a sequence comprising the nucleotides of SEQ ID NO: 1 containing nucleotides 168 to 173 and the other may be designed to have a sequence complementary to the nucleotides of SEQ ID NO: 1 at any other locations downstream of nucleotide 173, or a sequence comprising the nucleotides of SEQ ID NO: 3 containing nucleotides 120 to 125 and the other may be designed to have a sequence complementary to the nucleotides of SEQ ID NO: 3 at any other locations downstream of nucleotide 125. Alternatively, one of the primers may be designed to have a sequence complementary to the nucleotides of SEQ ID NO: 1 containing nucleotides 168 to 173 and the other may be designed to have a sequence comprising the nucleotides of SEQ ID NO: 1 at any locations upstream of 168, or a sequence complementary to the nucleotides of SEQ ID NO: 3 containing nucleotides 120 to 125 and the other may be designed to have a sequence comprising the nucleotides of SEQ ID NO: 3 at any locations upstream of 120. In this case, only ITPA1 and ITPA2 will be amplified.

Alternatively, one of the primers may be designed to have a sequence comprising the nucleotides of SEQ ID NO: 1 upstream of nucleotide 168 and the other may be designed to have a sequence complementary to the nucleotides of SEQ ID NO: 1 downstream of nucleotide 169, or a sequence comprising the nucleotides of SEQ ID NO: 3 upstream of nucleotide 122 and the other may be designed to have a sequence complementary to the nucleotides of SEQ ID NO: 3 downstream of nucleotide 123. Alternatively, one of the primers may be designed to have a sequence complementary to the nucleotides of SEQ ID NO: 1 upstream of nucleotide 168 and the other may be designed to have a sequence comprising the riucleotides of SEQ ID NO: 1 down stream of nucleotide 169, or a sequence complementary to the nucleotides of SEQ ID NO: 3 upstream of nucleotide 122 and the other may be designed to have a sequence comprising the nucleotides of SEQ ID NO: 3 downstream of nucleotide 123. In this case, ITPase gene, ITPA1 and ITPA2 will be amplified. The length of the PCR fragment from ITPA1 and ITPA2 will be 123 bp and 51 bp shorter than that from ITPase gene, respectively.

Preferably, the primer of the invention contains 15 to 30 nucleotides.

Total RNA may be isolated from patient samples by using TRIZOL reagents (Life Technology). Tissue samples (e.g., biopsy samples) are powdered under liquid nitrogen before homogenization. RNA purity and integrity are assessed by absorbance at 260/280 nm and by agarose gel electrophoresis. The set of primers designed to amplify the expected sizes of specific PCR fragments of ITPA1 and ITPA2 can be used. PCR fragments are analyzed on a 1% agarose gel using five microliters (10%) of the amplified products. To determine the expression levels for each gene variants, the intensity of the PCR products may be determined by using the Molecular Analyst program (version 1.4.1; Bio-Rad).

The RT-PCR experiment may be performed according to the manufacturer instructions (Boehringer Mannheim). A 50 μl reaction mixture containing 2 μl total RNA (0.1 μg/μl), 1 μl each primer (20 pM), 1 μl each dNTP (10 mM), 2.5 μDTT solution (100 mM), 10 ul 5X RT-PCR buffer, 1 μl enzyme mixture, and 28.5 μl sterile distilled water may be subjected to the conditions such as reverse transcription at 60° C. for 30 minutes followed by 35 cycles of denaturation at 94° C. for 2 minutes, annealing at 60° C. for 2 minutes, and extension at 68° C. for 2 minutes. The RT-PCR analysis may be repeated twice to ensure reproducibility, for a total of three independent experiments.

Another embodiment of the method for diagnosing the diseases associated with the deficiency of human ITPase gene may be performed by detecting the nucleotide sequences of ITPA1 and ITPA2, which comprises the steps of: (1) extracting total RNA from a sample obtained from the mammal; (2) amplifying the RNA by reverse transcriptase-polymerase chain reaction (RT-PCR) to obtain a cDNA sample; (3) bringing the cDNA sample into contact with the nucleic acid encoding the polypeptide comprising the amino acid sequences selected from the group consisting of SEQ ID NOS: 2 and 4 or the fragments thereof; and (4) detecting whether the cDNA sample hybridizes with the nucleic acid. If necessary, the amount of the hybridized sample may be determined.

The expression of gene variants can also be analyzed using Northern blot hybridization approach. Specific fragments comprising nucleotides 168 to 173 of ITPA1 and nucleotides 120 to 125 of ITPA2 may be amplified by polymerase chain reaction (PCR) using primer set designed for RT-PCR. The amplified PCR fragment may be labeled and serve as a probe to hybridize the membranes containing total RNAs extracted from the samples under the conditions of 55° C. in a suitable hybridization solution for 3 hr. Blots may be washed twice in 2×SSC, 0.1% SDS at room temperature for 15 minutes each, followed by two washes in 0.1×SSC and 0.1% SDS at 65° C. for 20 minutes each. After these washes, blot may be rinsed briefly in suitable washing buffer and incubated in blocking solution for 30 minutes, and then incubated in suitable antibody solution for 30 minutes. Blots may be washed in washing buffer for 30 minutes and equilibrated in suitable detection buffer before detecting the signals. Alternatively, the presence of gene variants (cDNAs or PCR) can be detected using microarray approach. The cDNAs or PCR products corresponding to the nucleotide sequences of the invention may be immobilized on a suitable substrate such as a glass slide. Hybridization can be preformed using the labeled mRNAs extracted from samples. After hybridization, nonhybridized mRNAs are removed. The relative abundance of each labeled transcript, hybridizing to a cDNA/PCR product immobilized on the microarray, can be determined by analyzing the scanned images.

According to the invention, the method for diagnosing the diseases associated with the deficiency of human ITPase gene may be performed by detecting the polypeptides encoded by the ITPA1 and ITPA2 of the invention. For instance, the polypeptide in protein samples obtained from the mammal may be determined by, but not limited to, the immunoassay wherein the antibodies specifically binding to the polypeptides of the invention is contacted with the sample, and the antibody-polypeptide complex is detected. If necessary, the amount of antibody-polypeptide complex can be determined.

The polypeptides encoded by the gene variants may be expressed in prokaryotic cells by using suitable prokaryotic expression vectors. The cDNA fragments of ITPA1 and ITPA2 genes encoding the amino acid coding sequence may be PCR amplified using primer set with restriction enzyme digestion sites incorporated in the 5′ and 3′ ends, respectively. The PCR products can then be enzyme digested, purified, and inserted into the corresponding sites of prokaryotic expression vector in-frame to generate recombinant plasmids. Sequence fidelity of this recombinant DNA can be verified by sequencing. The prokaryotic recombinant plasmids may be transformed into host cells (e.g., E. coli BL21 (DE3)). Recombinant protein synthesis may be stimulated by the addition of 0.4 mM isopropylthiogalactoside (IPTG) for 3 h. The bacterially-expressed proteins may be purified.

The polypeptides encoded by the ITPA1 and ITPA2 may be expressed in animal cells by using eukaryotic expression vectors. Cells may be maintained in Dulbecco's modified Eagle's medium (DMEM) supplemented with 10% fetal bovine serum (FBS; Gibco BRL) at 37° C. in a humidified 5% CO₂ atmosphere. Before transfection, the nucleotide sequence of each of the gene variant may be amplified with PCR primers containing restriction enzyme digestion sites and ligated into the corresponding sites of eukaryotic expression vector in-frame. Sequence fidelity of this recombinant DNA can be verified by sequencing. The cells may be plated in 12-well plates one day before transfection at a density of 5×10⁴ cells per well. Transfections may be carried out using Lipofectaminutese Plus transfection reagent according to the manufacturer's instructions (Gibco BRL). Three hours following transfection, medium containing the complexes may be replaced with fresh medium. Forty-eight hours after incubation, the cells may be scraped into lysis buffer (0.1 M Tris HCl, pH 8.0, 0.1% Triton X-100) for purification of expressed proteins. After these proteins are purified, monoclonal antibodies against these purified proteins (ITPA1 and ITPA2) may be generated using hybridoma technique according to the conventional methods (de StGroth and Scheidegger, (1980) J Immunol Methods 35:1-21; Cote et al. (1983) Proc Natl Acad Sci USA 80: 2026-30; and Kozbor et al. (1985) J Immunol Methods 81:31-42).

According to the invention, the presence of the polypeptides encoded by ITPA2 and ITPA2 in samples of normal lung and lung cancers may be determined by, but not limited to, Western blot analysis. Proteins extracted from samples may be separated by SDS-PAGE and transferred to suitable membranes-such as polyvinylidene difluoride (PVDF) in transfer buffer (25 mM Tris-HCl, pH 8.3, 192 mM glycine, 20% methanol) with a Trans-Blot apparatus for 1 h at 100 V (e.g., Bio-Rad). The proteins can be immunoblotted with specific antibodies. For example, membrane blotted with extracted proteins may be blocked with suitable buffers such as 3% solution of BSA or 3% solution of nonfat milk powder in TBST buffer (10 mM Tris-HCI, pH 8.0, 150 mM NaCl, 0.1% Tween 20) and incubated with monoclonal antibody specific to the polypeptides encoded by these gene variants. Unbound antibody is removed by washing with TBST for 5×1 minutes. Bound antibody may be detected using commercial ECL Western blotting detecting reagents.

The following examples are provided for illustration, but not for limiting the invention.

### EXAMPLES

### Analysis of Human Lung EST Databases

Expressed sequence tags (ESTs) generated from the large-scale PCR-based sequencing of the 5′-end of human lung (normal, SCLC, and large cell lung cancer) cDNA clones were compiled and served as EST databases. Sequence comparisons against the nonredundant nucleotide and protein databases were performed using BLASTN and BLASTX programs (Altschul et al., (1997) Nucleic Acids Res. 25: 3389-3402; Gish and States, (1993) Nat Genet 3:266-272), at the National Center for Biotechnology Information (NCBI) with a significance cutoff of p<10⁻¹⁰. ESTs representing putative ITPase gene encoding gene were identified during the course of EST generation.

### Isolation of cDNA Clones

Two cDNA clones exhibiting EST sequences similar to the ITPase gene were isolated from the lung cDNA libraries and named ITPA1 and ITPA2. The inserts of these clones were subsequently excised in vivo from the λZAP Express vector using the ExAssist/XLOLR helper phage system (Stratagene). Phagemid particles were excised by coinfecting XL1-BLUE MRF′ cells with ExAssist helper phage. The excised pBluescript phagemids were used to infect E. coli XLOLR cells, which lack the amber suppressor necessary for ExAssist phage replication. Infected XLOLR cells were selected using kanamycin resistance. Resultant colonies contained the double stranded phagemid vector with the cloned cDNA insert. A single colony was grown overnight in LB-kanamycin, and DNA was purified using a Qiagen plasmid purification kit.

### Full Length Nucleotide Sequencing and Database Comparisons

Phagemid DNA was sequenced using the Epicentre#SE9101LC SequiTherm EXCEL™II DNA Sequencing Kit for 4200S-2 Global NEW IR² DNA sequencing system (LI—COR). Using the primer-walking approach, fill-length sequence was determined. Nucleotide and protein searches were performed using BLAST against the non-redundant database of NCBI.

### In Silico Tissue Distribution Analysis

The coding sequence for each cDNA clones was searched against the dbEST sequence database (Boguski et al., (1993) Nat Genet. 4: 332-3) using the BLAST algorithm at the NCBI website. ESTs derived from each tissue were used as a source of information for transcript tissue expression analysis. Tissue distribution for each isolated cDNA clone was determined by ESTs matching to that particular sequence variants (insertions or deletions) with a significance cutoff of p<10⁻¹⁰.

### REFERENCES

Altschul et al., Gapped BLAST and PSI-BLAST: a new generation of protein database search programs, Nucleic Acids Res, 25: 3389-3402, (1997).
Ausubel et al., Current protocols in Molecular Biology, John Wiley & Sons, New York N.Y., ch. 9, 13, and 16, (1995).
Boguski et al., dbEST—database for “expressed sequence tags” . Nat Genet. 4: 332-3, (1993).
Carney, D. N. The biology of lung cancer. Curr. Opin. Oncol. 4: 292-8, (1992a).
Carney, D. N. Biology of small-cell lung cancer. Lancet 339: 843-6, (1992b).
Castonguay et al., Inhibition of lung tumorigenesis by NSAIDS: a working hypothesis. Exp Lung Res 24:605-15 (1998).
Cote et al., Generation of human monoclonal antibodies reactive with cellular antigens, Proc Natl Acad Sci USA 80: 2026-30 (1983).
de StGroth and Scheidegger, Production of monoclonal antibodies: strategy and tactics, J Immunol Methods 35:1-21, (1980).
Galperin, Conserved Hyperthetical proteins: new hints and new puzzles Comp Funct Genom 2: 14-18 (2001).
Gish and States, Identification of protein coding regions by database similarity search, Nat Genet, 3:266-272, (1993).
Hwang et al., Structure-based identification of a novel NTPase from Methanococcus jannaschii. Nat Struct Biol 6:691-6 (1999).
Ihde and Minna, Non-small cell lung cancer. Part II: Treatment. Curr. Probl. Cancer 15: 105-54, (1991).
Klinker and Seifert, Functionally nonequivalent interactions of guanosine 5′-triphosphate, inosine 5′-triphosphate, and xanthosine 5′-triphosphate with the retinal G-protein, transducin, and with Gi-proteins in HL-60 leukemia cell membranes. Biochem Pharmacol 54:551-62 (1997)
Kozak, An analysis of 5′-noncoding sequences from 699 vertebrate messenger RNAs. Nucleic Acids Res, 15: 8125-48, (1987).
Kozak, An analysis of vertebrate MRNA sequences: intimations of translational control, J Cell Biol, 115: 887-903, (1991).
Kozbor et al., Specific immunoglobulin production and enhanced tumorigenicity following ascites growth of human hybridomas, J Immunol Methods, 81:31-42 (1985).
Lin et al., Cloning, expression, and characterization of a human inosine triphosphate pyrophosphatase encoded by the itpa gene. J Biol Chem 276:18695-701 (2001).
Liu et al., Silent mutation induces exon skipping of fibrillin-1 gene in Marfan syndrome. Nat Genet 16:328-9, (1997).
Lukas et al., Alternative and aberrant messenger RNA splicing of the mdm2 oncogene in invasive breast cancer. Cancer Res 6-1:3212-9, (2001).
Michelland S, Gazzeri S, Brambilla E, Robert-Nicoud M. Comparison of chromosomal imbalances in neuroendocrine and non-small-cell lung carcinomas. Cancer Genet Cytogenet Oct. 1, 1999; 114(1):22-30
Roberge et al., A strategy for a convergent synthesis of N-linked glycopeptides on a solid support. Science 269:202-4, (1995).
Sambrook, J. Cold Spring Harbor Press, Plainview N.Y., ch. 4, 8, and 16-17.
Sethi, Science, medicine, and the future. Lung cancer, BMJ, 314: 652-655, (1997)
Siffert et al., Association of a human G-protein beta3 subunit variant with hypertension. Nat Genet, 18:45-8, (1998).
Smyth et al., The impact of chemotherapy on small cell carcinoma of the bronchus. Q J Med, 61: 969-76, (1986).
Stallings-Mann et al., Alternative splicing of exon 3 of the human growth hormone receptor is the result of an unusual genetic polymorphism. Proc Natl Acad Sci USA 93:12394-9, (1996).
Strausberg, R. EST Accession No. BI115811, and BG332818.
Vormittag W, Brannath W. As to the clastogenic-, sister-chromatid exchange inducing-and cytotoxic activity of inosine triphosphate in cultures of human peripheral lymphocytes. Mutat Res May 9, 2001; 476(1-2):71-81.

1. An isolated polypeptide comprising the amino acid sequences selected from the group consisting of SEQ ID NOS: 2 and 4, and fragments thereof.

2. The isolated polypeptide of claim 1, wherein the fragments comprise the amino acid residues 22 to 23 of SEQ ID NO: 2.

3. The isolated polypeptide of claim 1, wherein the fragments comprise the amino acid residues 6 to 7 of SEQ ID NO: 4.

4. An isolated nucleic acid encoding a polypeptide selected from the group consisting of SEO ID NOS: 2 and 4, and fragments thereof.

5. The isolated nucleic acid of claim 4, which comprises the nucleotide sequence selected from the group consisting of SEQ ID NOS: 1 and 3.

6. The isolated nucleic acid of claim 4, wherein the fragments comprise nucleotides 168 through 173 of SEQ ID NO: 1.

7. The isolated nucleic acid of claim 4, wherein the fragments comprise nucleotides 120 through 125 of SEQ ID NO: 3.

8. An expression vector comprising the nucleic acid of claim 4.

9. A host cell transformed with the expression vector of claim 8.

10. A method for producing an isolated polypeptide comprising the amino acid sequences selected from the group consisting of SEQ ID NOS: 2 and 4, and fragments thereof, which comprises the steps of:
(1) culturing the host cell of claim 9 under a condition suitable for the expression of the polypeptide; and
(2) recovering the polypeptide from the host cell culture.

11. An antibody specifically binding to the polypeptide of claim 1.

12. A method for diagnosing the diseases associated with the nucleic acid of claim 4, in particular lung cancers, in a mammal which comprises detecting an isolated nucleic acid encoding an isolated polypeptide comprising the amino acid sequences selected from the group consisting of SEQ ID NOS: 2 and 4, and fragments thereof or an isolated polypeptide comprising the amino acid sequences selected from group consisting of SEQ ID NOS: 2 and 4. and fragments thereof.

13. The method of claim 12, wherein the detection of an isolated nucleic acid encoding an isolated polvpeptide comprising the amino acid sequences selected from the group consisting of SEQ ID NOS: 1 and 4, and fragments thereof comprises the steps of:
(1) extracting total RNA from a sample obtained from the mammal;
(2) amplifying the RNA by reverse transcriptase-polymerase chain reaction (RT-PCR) to obtain a cDNA sample;
(3) bringing the cDNA sample into contact with an isolated nucleic acid encoding an isolated polypeptide comprising the amino acid sequences selected from the group consisting of SEQ ID NOS: 2 and 4, and fragments thereof and
(4) detecting whether the cDNA hybridizes with an isolated nucleic acid encoding an isolated polvpeptide comprising the amino acid sequences selected from the group consisting of SEQ ID NOS: 2 and 4, and fragments thereof.

14. The method of claim 13 further comprising the step of determining the amount of hybridized sample.

15. The method of claim 12, wherein the detection of the an isolated polypeptide comprising the amino acid sequences selected from the group consisting of SEO ID NOS: 2 and 4, and fragments thereof comprises the steps of contacting an antibody specifically binding to an isolated polypeptide comprising the amino acid sequences selected from the group consisting of SEO ID NOS: 2 and 4, and fragments thereof with protein samples extracted from the mammal, and detecting whether an antibody-polypeptide complex is formed.

16. The method of claim 15 further comprising the step of determining the amount of the antibody-polypeptide complex.

17. The method of claim 12, wherein the detection of the an isolated nucleic acid encoding an isolated polypeptide comprising the amino acid sequences selected from the group consisting of SEO ID NOS: 2 and 4, and fragments thereof comprises the steps of:
(1) extracting the total RNAs of cells obtained from the mammal;
(2) amplifying the RNA by reverse transcriptase-polymerase chain reaction (RT-PCR) with a set of primers to obtain a cDNA comprising the fragments comprising nucleotide 168 through 173 of SEQ ID NO: 1 or nucleotide 120 through 125 of SEQ ID NO: 3; and
(3) detecting whether the cDNA sample is obtained.

18. The method of claim 17, wherein one of the primers has a sequence comprising the nucleotides of SEQ ID NO: 1 containing nucleotides 168 to 173, and the other has a sequence complementary to the nucleotides of SEQ ID NO: 1 at any other locations downstream of nucleotide 173, or one of the primers has a sequence complementary to the nucleotides of SEQ ID NO: 1 containing nucleotides 168 to 173, and the other has a sequence comprising the nucleotides of SEQ ID NO: 1 at any other locations up stream of nucleotide 168.

19. The method of claim 17, wherein one of the primers has a sequence comprising the nucleotides of SEQ ID NO: 3 containing nucleotides 120 to 125, and the other has a sequence complementary to the nucleotides of SEQ ID NO: 3 at any other locations downstream of nucleotide 125, or one of the primers has a sequence complementary to the nucleotides of SEQ ID NO: 3 containing nucleotides 120 to 125, and the other has a sequence comprising the nucleotides of SEQ ID NO: 3 at any other locations up stream of nucleotide 120.

20. The method of claim 17, wherein one of the primers has a sequence comprising the nucleotides of SEQ ID NO: 1 upstream of nucleotide 168 and the other has a sequence complementary to the nucleotides of SEQ ID NO: 1 downstream of nucleotide 169, or one of the primers has a sequence complementary to the nucleotides of SEQ ID NO: 1 upstream of nucleotide 168 and the other has a sequence comprising the nucleotides of SEQ ID NO: 1 downstream of nucleotide 169.

21. The method of claim 20, wherein the cDNA sample amplified from SEQ ID NO: 1 is 123 bp shorter than the cDNA sample amplified from human ITPase.

22. The method of claim 17, wherein one of the primers has a sequence comprising the nucleotides of SEQ ID NO: 3 upstream of nucleotide 122 and the other has a sequence complementary to the nucleotides of SEQ ID NO: 3 downstream of nucleotide 123, or one of the primers has a sequence complementary to the nucleotides of SEQ ID NO: 3 upstream of nucleotide 122 and the other has a sequence comprising the nucleotides of SEQ ID NO: 3 downstream of nucleotide 123.

23. The method of claim 22, wherein the cDNA sample amplified from SEQ ID NO: 3 is 51 bp shorter than the cDNA sample amplified from human ITPase.

24. The method of claim 17 further comprising the step of determining the amount of the amplified cDNA sample.
